(19) United States
(12) Patent Application Publication
(10) Pub. No.: US 2020/0276217 A1
(43) Pub. Date: Sep. 3, 2020

(54) Application of Macrolide Antibiotic in Blocking Influenza Virus Infection

(71) Applicant: SUZHOU INSTITUTE OF SYSTEMS MEDICINE, Suzhou (CN)

(72) Inventors: Frank-Xiaofeng QIN, SUZHOU (CN); Xiangyang ZUO, SUZHOU (CN); Xiaohong DU, SUZHOU (CN)

(73) Assignee: SUZHOU INSTITUTE OF SYSTEMS MEDICINE, Suzhou (CN)

(21) Appl. No.: 16/880,414

(22) Filed: May 21, 2020

Related U.S. Application Data
(63) Continuation of application No. PCT/CN2018/089631, filed on Jun. 1, 2018.

(30) Foreign Application Priority Data
Nov. 24, 2017 (CN) 201711194999.8

(51) Int. Cl. A61K 31/7052; A61K 31/13; A61K 31/215; A61K 31/351; A61P 31/16

(52) U.S. Cl. CPC A61K 31/7052; A61K 31/13; A61K 31/7048; A61K 31/351; A61P 31/16; A61K 31/215

(57) ABSTRACT

Provided are use of a macrolide antibiotic in blocking influenza virus infection and use of the macrolide antibiotic in preparing a drug for treating or preventing influenza virus infection.

### CROSS-REFERENCE TO EARLIER FILED APPLICATIONS

This application claims the benefit of and is a continuation of PCT/CN2018/089631, filed Jun. 1, 2018, which claims the benefit of CN 201711194999.8, filed Nov. 24, 2017, the entire disclosures of which are hereby incorporated by reference.

### TECHNICAL FIELD OF THE INVENTION

The present disclosure relates to the use of a class of antibiotics and a composition comprising the aforementioned antibiotics in the preparation of a drug for preventing and treating a disease, specifically, relates to the use of a macrolide antibiotic or a pharmaceutically acceptable salt thereof and a composition comprising the aforementioned antibiotic or the pharmaceutically acceptable salt thereof in the preparation of a drug for blocking influenza virus infection.

### BACKGROUND OF THE INVENTION

Influenza virus is a pathogen which seriously threatens human health and life safety, and may be divided into three types (i.e., influenza A virus, influenza B virus and influenza C virus). Among them, the antigenicity of the influenza A virus mutates easily and the influenza A virus has caused pandemics for many times. For instance, the swine-derived H1N1 influenza virus infected human and spread widely among the population in 2009, resulting in the first influenza pandemic in the 21^{st} century. Laboratory-diagnosed virus infection alone resulted in a death toll of up to 18500 around the world, which caused great harm to human life health and economic property^{[1-4]}. Currently, the methods used for resisting an influenza virus are mainly vaccination or drug treatment. The drugs mainly comprise amantadine and rimantadine targeting the ion channel protein (M2) of an influenza virus, oseltamivir and zanamivir targeting a surface protein (NA), and the like; while the design of the related vaccines also takes hemagglutinin (HA) and neuraminidase (NA) as target antigens to induce the generation of immune protection in the body. However, since the RNA polymerase of an influenza virus lacks fidelity, the influenza virus, especially the surface protein(s) thereof, mutates particularly fast. Therefore, there are some problems in the use of the existing vaccines and small molecule drugs targeting the surface protein(s) of an influenza virus, for example, the vaccine mismatch, the occurrence of resistant viruses, and the like. Moreover, since the numerous subtypes of influenza viruses, the wide range of hosts, the infection of human through the passage of species barriers, the occurrence of a highly pathogenic avian influenza virus and the like have brought extremely great difficulties to the prevention and control of influenza viruses^{[5-9]}, there is a need to develop more effective methods to suppress the replication and spread of influenza viruses.

New use of pre-existing drugs means that known drugs are used for treating new indications. In recent years, with the extensive research conducted by scientists, they often accidentally discover that some drugs that have been used for many years may probably be capable of treating a variety of other human diseases^{[10-17].} For example, the pre-existing drug flavopiridol may be used as an effective therapy to cut off the short-chain sugar molecules utilizable for cancer cells and impair the growth of cancer cells effectively^{[10]}. It has also been reported that the antidiabetic drug metformin may inhibit the growth of pancreatic cancer cells ^{[11]}, while tamoxifen used for treating advanced breast cancer and ovarian cancer has been proved to be capable of exerting quite strong inhibitory effect in experiments of resisting Ebola virus infection^{[15]}. In addition, researchers have also found in the antiviral treatment that tetrandrine used for the treatment of severe hypertension may be considered as a nemesis of Ebola and is expected to be used in the treatment of diseases caused by Ebola virus infection within the next two to five years^{[17]}. Since the original indications, in vitro activity, clinical dosage and toxic and side effects of the listed drugs have been clarified, once proved to have good therapeutic effects on other indications, these drugs may be put into use quickly, thus greatly shortening the research and development process of new drugs.

Macrolide antibiotics refer to broad-spectrum antibiotics produced by streptomyces. Macrolide antibiotics have basic lactone ring structures, are effective against Gram-positive bacteria and Gram-negative bacteria, and particularly have relatively strong effects on mycoplasma, chlamydia, legionella, spirochete and rickettsia. Macrolide antibiotics may be classified into 14- to 16-membered macrolide antibiotics according to the different number of carbon atoms contained in the parent nucleuses of the lactone structures. Listed macrolide antibiotic are mainly classified into three categories, that is, erythromycins, midecamycins and spiramycins. Erythromycins and the (ester) derivatives thereof (such as erythromycin ethylsuccinate, erythromycin estolate, roxithromycin, clarithromycin, dirithromycin and Flurithromycin) belong to 14-membered macrolide antibiotics, while azithromycin derived from erythromycin is the first 15-membered azamacrolide (azalide) antibiotic launched in the market, and midecamycins and the derivatives thereof as well as spiramycins and the derivatives thereof (for example, acetyl spiramycin) belong to 16-membered macrolides.

Macrolide antibiotics are capable of irreversibly binding to the 50S subunit of the bacterial ribosome and selectively inhibiting protein synthesis by blocking transpeptidation and mRNA translocation. A macrolide antibiotic may bind to a specific target site (i.e., 23S rRNA in 50S subunit) to prevent the peptidyl-tRNA from translocating from “A” site to “P” site on mRNA and disable the aminoacyl-tRNA from binding to “A” site, thus selectively inhibiting the synthesis of bacterial proteins; or may bind to the L22 protein of the 50S subunit of the bacterial ribosome, cause the disruption of the ribosomal structure, and allow the peptidyl-tRNA to dissociate from the ribosome relatively early in the elongation stage of peptide chain.

Azithromycin is a 15-membered macrolide antibiotic. By binding to the 50s subunit of the bacterial ribonucleoprotein, this drug prevents the elongation of peptide chain and affects the synthesis of bacterial proteins to achieve bacteriostatic effect, and is effective against the majority of the Gram-positive bacteria, some of the Gram-negative bacteria and some atypical pathogenic bacteria. Azithromycin is the drug with the strongest bacteriostatic activity against Gram-negative cocci such as gonococcus and meningococcus among macrolide antibiotics, and is often used for tonsillitis, pneumonia, urethritis and the like caused by bacterial infection^{[18]}.

However, there is no report on the direct inhibition of influenza virus infection caused by a macrolide antibiotic as an active ingredient in the existing prior art related to macrolide antibiotics.

### SUMMARY OF THE INVENTION

The object of the present disclosure is to provide the use of macrolide antibiotics including azithromycin, erythromycin, roxithromycin, midecamycin or acetylspiramycin or a pharmaceutically acceptable salt thereof and a composition comprising the aforementioned antibiotics in the preparation of a drug for blocking influenza virus infection.

Another object of the present disclosure is to provide a composition for preventing or treating influenza virus infection, and the composition comprises a macrolide antibiotic including azithromycin, erythromycin, roxithromycin, midecamycin or acetylspiramycin as an active ingredient for suppressing an influenza virus.

Specifically, the present disclosure is an evaluation of the anti-influenza effects of a variety of small molecule drugs that have been launched in the market and clinically used, based on the IAV-luc model prepared by the technique of the reverse genetic manipulation of influenza virus^{[19]}. It has been found that macrolide antibiotics including azithromycin, erythromycin, roxithromycin, midecamycin and acetylspiramycin have great anti-influenza virus activities in vitro, and studies conducted in mice have also shown that the macrolide antibiotics including azithromycin may prophylactically treat the infection of influenza. It has been revealed by further studies that macrolide antibiotics including azithromycin may directly act on the virus particles so as to block the infection of an influenza virus.

In order to solve the technical problems of the present disclosure, the present disclosure provides the following technical solutions.

First, the present disclosure provides the use of a macrolide antibiotic or a pharmaceutically acceptable salt thereof as an active component for inhibiting an influenza virus infection in the preparation of a drug for treating or preventing influenza virus infection.

In the use according to the present disclosure, the antibiotic is selected from one or more of azithromycin, erythromycin, roxithromycin, midecamycin and acetylspiramycin.

In the use according to the present disclosure, the antibiotic is selected from azithromycin.

In the use according to the present disclosure, the type of the influenza virus comprises an influenza A virus, an influenza B virus, an influenza C virus, or a combination thereof, preferably, the type of the influenza virus is the influenza A virus, and more preferably, the type of the influenza virus is an influenza A H1N1 virus.

In the use according to the present disclosure, the drug further comprises a therapeutically effective amount of an antiviral antibiotic.

In the use according to the present disclosure, the antiviral antibiotic comprises one or more selected from amantadine, rimantadine, oseltamivir and zanamivir.

In the use according to the present disclosure, the drug further comprises one or more pharmaceutically acceptable carriers.

In the use according to the present disclosure, the drug or a pharmaceutically acceptable salt thereof is administered via one of the following routes: intravenous administration, intraperitoneal administration, intracoronary administration, intraarterial administration, intradermal administration, subcutaneous administration, transdermal delivery, intratracheal administration, intraarticular administration, intraventricular administration, inhalation, intracerebral administration, transumbilical administration, oral administration, intraocular administration, pulmonary administration, injection via a catheter, administration via a suppository, and direct injection into a tissue.

In some embodiments, the antibiotic according to the present disclosure or a pharmaceutically acceptable salt thereof is administered systemically. “Systemic administration” as used in the present disclosure refers to any means by which the antibiotic according to the present disclosure is enabled to be available systemically. In some embodiments, systemic administration encompasses intravenous administration, intraperitoneal administration, intramuscular administration, intracoronary administration, intraarterial administration (for example, administration into carotid artery), intradermal administration, subcutaneous administration, transdermal delivery, intratracheal administration, subcutaneous administration, intraarticular administration, intraventricular administration, inhalation (for example, aerosol), intracerebral administration, nasal administration, transumbilical administration, oral administration, intraocular administration, pulmonary administration, injection via a catheter, administration via a suppository, and direct injection into a tissue, or local or mucosal administration with systemic absorption. Mucosal administration comprises administration to respiratory tract tissues, for example, via inhalation, a nasal drop, an eye drop, and the like; the administration routes via anus or vagina, for example, administration via a suppository; and similar administration routes. In some embodiments, the antibiotic according to the present disclosure or a pharmaceutically acceptable salt thereof is administered intravenously. In other embodiments, the antibiotic according to the present disclosure or a pharmaceutically acceptable salt thereof is administered orally. In some embodiments, the antibiotic according to the present disclosure or a pharmaceutically acceptable salt thereof may be administered intravenously once to five times per week. In some other embodiments, the antibiotic according to the present disclosure or a pharmaceutically acceptable salt thereof may be administered orally once or more times a day (for example, once a day, twice a day, or three times a day).

The present disclosure also provides a composition for preventing or treating influenza virus infection, wherein the composition comprises a macrolide antibiotic or a pharmaceutically acceptable salt thereof as a component for inhibiting viral infection, preferably, the antibiotic is selected from one or more of azithromycin, erythromycin, roxithromycin, midecamycin and acetylspiramycin; and more preferably, the antibiotic is selected from azithromycin.

In some embodiments, the pharmaceutical composition provided by the present disclosure optionally comprises a pharmaceutical carrier, wherein the pharmaceutical carrier refers to a pharmaceutical carrier commonly used in the pharmaceutical field. This pharmaceutical composition may be prepared according to a method well known in the art. Any dosage form suitable for human or animal use may be prepared by combining the antibiotic of the present disclosure or a pharmaceutically acceptable salt thereof with one or more pharmaceutically acceptable solid or liquid excipients and/or adjuvants.

A dosage form for administration may be a liquid dosage form, a solid dosage form or a semi-solid dosage form. The liquid dosage form may be a solution (including a true solution and a colloidal solution), an emulsion (including an O/W-type emulsion, a W/O-type emulsion and a multiple emulsion), a suspension, an injection (including an aqueous injection, a powder for injection and an infusion), an eye drop, a nasal drop, a lotion, a liniment, and the like; the solid dosage form may be a tablet (including a conventional tablet, an enteric coated tablet, a buccal tablet, a dispersible tablet, a chewable tablet, an effervescent tablet, an oral disintegrating tablet), a capsule (including a hard capsule, a soft capsule, an enteric capsule), a granule, a powder, a pellet, a dropping pill, a suppository, a film, a patch, a gas (powder) aerosol, a spray, and the like; and the semi-solid dosage form may be an ointment, a gel, a paste, and the like.

The antibiotic of the present disclosure or a pharmaceutically acceptable salt thereof may be prepared into an ordinary preparation, and may also be prepared into a sustained-release preparation, a controlled-release preparation, a targeting preparation and various microparticle delivery systems.

In order to prepare the antibiotic and the pharmaceutically acceptable salt thereof of the present disclosure into a tablet, it is possible to widely use various excipients well known in the art, including a diluent, a binder, a wetting agent, a disintegrating agent, a lubricant, and a glidant. The diluent may be starch, dextrin, sucrose, glucose, lactose, mannitol, sorbitol, xylitol, microcrystalline cellulose, calcium sulfate, calcium hydrogen phosphate, calcium carbonate, etc.; the wetting agent may be water, ethanol, isopropanol, etc.; the binder may be starch slurry, dextrin, syrup, honey, glucose solution, microcrystalline cellulose, mucilage of arabic gum, gelatin mucilage, sodium carboxymethyl cellulose, methyl cellulose, hydroxypropyl methylcellulose, ethyl cellulose, acrylic resin, carbomer, polyvinylpyrrolidone, polyethylene glycol, etc.; the disintegrating agent may be dry starch, microcrystalline cellulose, low-substituted hydroxypropyl cellulose, cross-linked polyvinylpyrrolidone, cross-linked sodium carboxymethyl cellulose, sodium carboxymethyl starch, sodium bicarbonate and citric acid, polyoxyethylene sorbitol fatty acid ester, sodium dodecyl sulfonate, etc.; and the lubricant and the glidant may be talc, silica, stearate, tartaric acid, liquid paraffin, polyethylene glycol, etc.

It is also possible to further prepare a tablet into a coated tablet, for example, a sugar-coated tablet, a film-coated tablet, an enteric-coated tablet, or into a double-layer tablet or a multi-layer tablet.

In order to prepare a dosage unit into a capsule, it is possible to mix a therapeutically effective amount of the antibiotic of the present disclosure or a pharmaceutically acceptable salt thereof with a diluent and/or a glidant, and the mixture is directly placed in a hard capsule or a soft capsule. A therapeutically effective amount of the antibiotic of the present disclosure or a pharmaceutically acceptable salt thereof together with a diluent, a binder and/or a disintegrating agent may also be first prepared into a granule or a pellet, which is then placed in a hard capsule or a soft capsule. Each of the diluents, binders, wetting agents, disintegrating agents and glidants used for preparing the antibiotic of the present disclosure or a pharmaceutically acceptable salt thereof may also be used to prepare a capsule of the antibiotic of the present disclosure or a pharmaceutically acceptable salt thereof.

In order to prepare the antibiotic of the present disclosure or a pharmaceutically acceptable salt thereof into an injection, water, ethanol, isopropanol, propylene glycol or a mixture thereof may be used as a solvent, and a proper amount of a solubilizer, a co-solvent, a pH adjusting agent and/or an osmotic pressure regulator commonly used in the art are added. The solubilizer or the co-solvent may be poloxamer, lecithin, hydroxypropyl-β-cyclodextrin, etc.; the pH adjusting agent may be phosphate, acetate, hydrochloric acid, sodium hydroxide, etc.; the osmotic pressure regulator may be sodium chloride, mannitol, glucose, phosphate, acetate, etc. If a lyophilized powder for injection is to be prepared, mannitol, glucose, and the like may also be added as a proppant.

In addition, a colorant, a preservative, a perfume, a flavoring agent or other additives may be added to a pharmaceutical preparation if necessary.

In order to achieve the purpose of drug administration and enhance the therapeutic effect, the drug or the pharmaceutical composition of the present disclosure may be administered via any well-known administration method.

### Effects of the Present Disclosure

It is confirmed by the present disclosure that macrolide antibiotics or the pharmaceutically acceptable salts thereof have good inhibitory effects against influenza viruses. Among them, the inhibitory effect of azithromycin against influenza viruses is superior to that of erythromycin, roxithromycin, midecamycin or acetylspiramycin; meanwhile, azithromycin exerts the antiviral ability mainly by acting on virus particles. Therefore, the aforementioned macrolide antibiotic or a pharmaceutically acceptable salt thereof and the composition comprising the aforementioned macrolide antibiotic may be prepared into a drug for treating or preventing influenza virus infection.

### BRIEF DESCRIPTION OF THE DRAWINGS

FIG. 1 shows the comparison between the efficacies of macrolide antibiotics against influenza virus infection, wherein each macrolide antibiotic has a concentration of 10 μM.

FIG. 2 shows the screening and evaluation of anti-influenza virus small molecule compounds, and each small molecule compound has a concentration of 10 μM. The drugs in the figure are numbered as follows. 1: absolute ethyl alcohol, 2: tamoxifen, 3: azithromycin, 4: fluvastatin, 5: amiodarone, 6: fluoxetine, 7: clemastine, 8: delphinidin, 9: chloroquine, 10: amlodipine besylate, 11: ribavirin.

FIG. 3 shows the comparison between the inhibitory effects of azithromycin and oseltamivir against influenza virus infection, wherein azithromycin and oseltamivir both have a concentration of 10 μM.

FIG. 4 shows the comparison between the protective effects of azithromycin and ribavirin on the lungs of mice infected with an influenza virus, wherein the type of the influenza virus is A/WSN33/H1N1 influenza virus.

FIG. 5 shows the inhibitory effects of different concentrations of azithromycin against influenza virus infection, and an HA/NA-HIVLuc pseudovirus system capable of mimicing the influenza virus infection is adopted.

FIG. 6 shows the effects of azithromycin and ribavirin on the replication of the influenza virus polymerase.

FIG. 7 shows that azithromycin exerts its anti-influenza virus effect mainly by directly acting on the virus particles, wherein the adopted cell line is A549 cell line.

### DETAILED DESCRIPTION OF THE INVENTION

Unless otherwise defined, the technical and scientific terms used in the present disclosure have the same meanings as commonly understood by those of ordinary skill in the technical field to which the present disclosure pertains.

It should be understood that, unless explicitly stated otherwise, all values and ranges falling within the listed numerical values and numerical ranges are intended to be encompassed by the present disclosure.

### Definitions

The term “macrolide antibiotic” is a general term for a class of antibacterial antibiotics with a 14- to 16-membered lactone ring in their molecular structures, and a macrolide antibiotic inhibits bacterial protein synthesis by blocking the activity of the peptidyl transferase in 50s ribosome.

The term “effective amount” refers to an amount of a compound which is sufficient to provide the desired effect with no toxicity or acceptable toxicity. This amount may vary among different subjects, depending on the species, age and physical condition of the subject, the severity of the disease being treated, the specific compound used and the administration mode thereof, and the like. An appropriate effective amount may be determined by those of ordinary skill in the art.

“Treating” as used herein is defined as applying or administering a therapeutic agent to a subject or an isolated tissue or cell line derived from the subject. Generally, said subject suffers from a disease or disorder, has a symptom of the disease or disorder, or is susceptible to the disease or disorder (for example, influenza). To be specific, the treatment as used herein aims at a subject that has already been infected with a virus (such as influenza) instead of a subject that has not been infected. In general, the purpose of the treatment is to cure, heal, mitigate, alleviate, remedy, relieve or ameliorate said disease, disorder or symptom. “Treated” as used herein refers to that the disease or disorder is cured, healed, mitigated, alleviated, remedied, relieved or ameliorated.

An antibiotic suitable for use in the methods of the present disclosure is administered in a therapeutically effective dosage. The term “therapeutically effective amount” is used to refer to the amount of an active antibiotic or a pharmaceutical preparation that causes the indicated biological response or drug response. Such response may appear in the tissues and systems (animals including humans) that are intended to be treated by researchers, veterinarians, physicians or other clinicians.

The chemical structural formula of “azithromycin” referred to in the present disclosure is as follows.

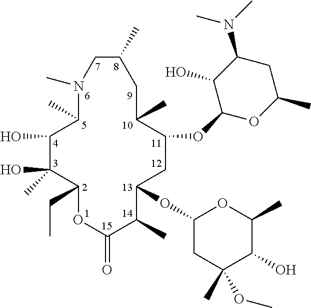 [SMILES: CC[C@H]1OC(=O)[C@H](C)[C@@H](O[C@H]2C[C@@](C)(OC)[C@@H](O)[C@H](C)O2)C[C@@H](O[C@@H]2O[C@H](C)C[C@H](N(C)C)[C@H]2O)[C@@H](C)C[C@@H](C)CN(C)[C@H](C)[C@@H](O)[C@]1(C)O]

The chemical structural formula of “erythromycin” referred to in the present disclosure is as follows.

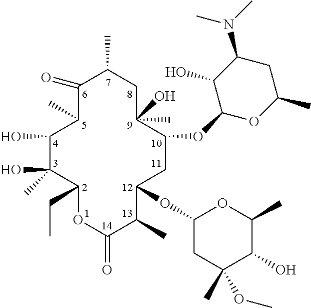 [SMILES: CC[C@H]1OC(=O)[C@H](C)[C@@H](O[C@H]2C[C@@](C)(OC)[C@@H](O)[C@H](C)O2)C[C@@H](O[C@@H]2O[C@H](C)C[C@H](N(C)C)[C@H]2O)[C@](C)(O)C[C@@H](C)C(=O)[C@H](C)[C@@H](O)[C@]1(C)O]

The chemical structural formula of “roxithromycin” referred to in the present disclosure is as follows.

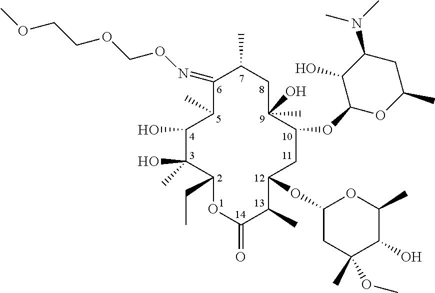

The chemical structural formula of “midecamycin” referred to in the present disclosure is as follows.

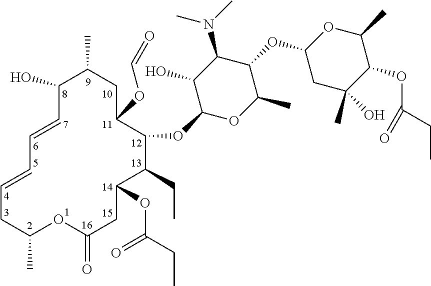 [SMILES: CCC(=O)O[C@@H]1CC(=O)O[C@H](C)C/C=C/C=C/[C@H](O)[C@H](C)C[C@H](OC=O)[C@H](O[C@@H]2O[C@H](C)[C@@H](O[C@H]3C[C@@](C)(O)[C@@H](OC(=O)CC)[C@H](C)O3)[C@H](N(C)C)[C@H]2O)[C@H]1CC]

The chemical structural formula of “acetylspiramycin” referred to in the present disclosure is as follows.

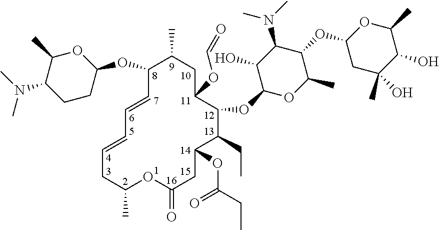 [SMILES: CCC(=O)O[C@@H]1CC(=O)O[C@H](C)C/C=C/C=C/[C@H](O[C@H]2CC[C@H](N(C)C)[C@@H](C)O2)[C@H](C)C[C@H](OC=O)[C@H](O[C@@H]2O[C@H](C)[C@@H](O[C@H]3C[C@@](C)(O)[C@@H](O)[C@H](C)O3)[C@H](N(C)C)[C@H]2O)[C@H]1CC]

### EXAMPLES

Various exemplary examples, features, and aspects of the present disclosure will be described in detail below with reference to the attached drawings. The wording “exemplary” specifically used herein means “being used as an example or an embodiment, or illustration”. Any example described herein as an “exemplary” example is not necessary to be interpreted as superior to or better than other examples.

In addition, in order to better illustrate the present disclosure, numerous specific details are given in the specific embodiments hereinafter. Those skilled in the art should understand that the present disclosure may also be implemented without certain specific details. In some other examples, methods, means, apparatus and steps well known to those skilled in the art are not described in detail, so as to highlight the gist of the present disclosure.

### Example 1: Macrolide Antibiotics had the Ability to Resist Influenza a Virus A/Puerto Rico/8/34(PR8)-Luc Infection

A549 cell (human lung cancer epithelial cell) was a relatively ideal cell line for the research of an influenza virus. The inventors evaluated the abilities of several small molecule antibiotics that had been launched in the market (including 14-membered macrolide antibiotics erythromycin and roxithromycin, 15-membered macrolide antibiotic azithromycin, and 16-membered macrolide antibiotics acetylspiramycin and midecamycin) to resist influenza virus infection by using an influenza virus model of IAV-Luc carrying the luciferase reporter gene (the influenza virus PR8 was used as a backbone, and a humanized luciferase gene was introduced to the C-terminus of the NA-encoding gene) which was constructed via a reverse genetic system by Chen Ling's team in the Chinese Academy of Sciences. The inventors first seeded A549 cells in a 96-well plate with 10000 cells per well, and the cells were incubated with a drug after 16 hours. After 8 hours, the influenza virus IAV-Luc was added to infect the cells, and the concentration of the virus inoculum was 10⁶ RLU/CELL. 24 hours later, the supernatant was collected to conduct luciferase reporter gene detection, and the reading of the reporter gene reflected the condition of influenza virus infection. The inventors used absolute ethyl alcohol as a negative control group, and the infection rates of influenza virus of all administration groups were results relative to the result of the negative control group using absolute ethyl alcohol.

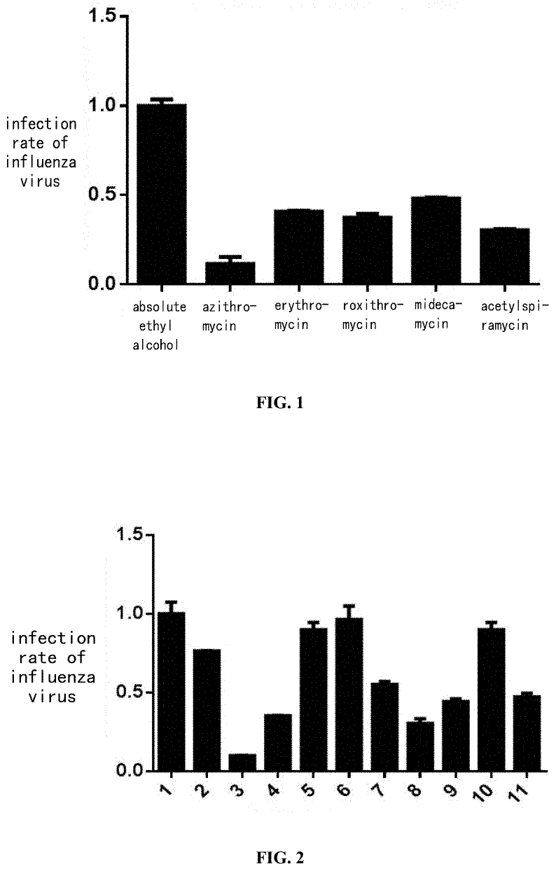

After data analysis, as shown in FIG. 1, it was shown that macrolide antibiotics such as azithromycin, erythromycin, roxithromycin, midecamycin or acetylspiramycin had the ability to resist influenza virus infection. Among them, azithromycin had the strongest efficacy. Meanwhile, as shown in FIG. 2, azithromycin had quite strong inhibitory effect against influenza virus infection.

### Example 2: Comparison Between the Abilities of Azithromycin and Oseltamivir to Resist Influenza Virus Infection

Since azithromycin was often used as an adjuvant when oseltamivir was used to treat an influenza virus in the prior art, the abilities of azithromycin and oseltamivir to resist influenza virus infection were compared separately. The abilities of azithromycin and oseltamivir to resist influenza virus infection were compared by using the same experimental methods and steps as in Example 1.

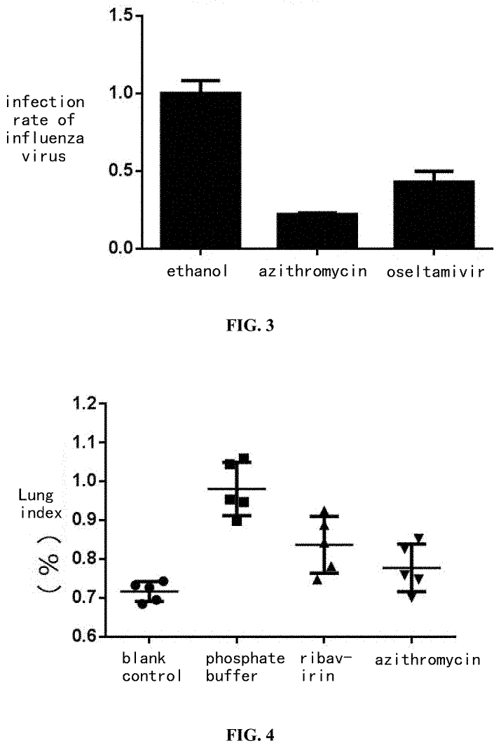

As shown in FIG. 3, after data analysis, it was found that the ability of azithromycin to resist influenza virus infection was stronger than that of oseltamivir at a same concentration (10 μM).

### Example 3: Azithromycin could be Used for the Prophylactic Treatment of A/WSN33/H1N1 in Barb/C Mice

24 female Barb/C mice (6- to 8-week old) were randomly divided into 4 groups (A, B, C, D) with 5 mice in each group. All animals were marked by ear clipping and then treated with a specified preparation. Group A was a blank control group, Group B was a group in which PBS was used to resist virus infection, Group C was a group in which ribavirin was used to resist virus infection, and Group D was a group in which azithromycin was used to resist virus infection. Two days before the drugs being used to resist virus infection, the mice were treated with 50 mg/kg ribavirin and 50 mg/kg azithromycin by intragastric administration, and were infected with the influenza virus via nasal dripping with a dose of 40 LD₅₀ (10³ PFU/ml) on the third day (50 μl/mouse), and PBS was administered instead in the blank control group. The mice were administered consecutively for 5 days after virus infection, and the administration method was same as above. After 12 hours of fasting and water deprivation, the body weights were weighed, the lungs were incised aseptically and weighed after the mice were sacrificed by eyeball extirpating, and the lung indexes were calculated so as to evaluate the protective effect of azithromycin for mice in influenza virus infection, in which the calculation method of the lung index was a conventional calculation method known in the art.

FIG. 4 showed the protective effect of azithromycin for mice in influenza virus infection, proving that azithromycin had better protective effect for mice in influenza virus infection as compared with ribavirin.

### Example 4: Azithromycin Inhibited Influenza Virus Infection and the Inhibitory Effect was Concentration-Dependent

In order to further study the mechanism of the inhibition of influenza virus exerted by azithromycin, an HA/NA-HIVLuc pseudovirus system that could mimic influenza virus infection and a polymerase system (Flu-RNA polymerase) used for detecting the stage of transcription and replication were used. First, A549 cells were seeded in a 96-well plate with 10000 cells per well, and cells were pre-incubated with azithromycin of various concentration gradients after 16 hours. After 8 hours, a pseudo-influenza virus was added to infect the cells, the medium was replaced after 24 hours, the detection was carried out after another 48 hours, the supernatant was discarded, and then, to the infected cells, 50 μl of the cell lysis buffer were added per well. 25 μl of the luciferase substrate (Promega) was mixed with 25 μl of the cell lysis buffer, then the relative activity of luciferase was detected, and its activity reflected the extent of virus infection. The inventors still used absolute ethyl alcohol as a negative control group, and the infection rates of influenza virus in administration groups administered with drug of various concentration gradients were all results relative to the result of the absolute ethyl alcohol group.

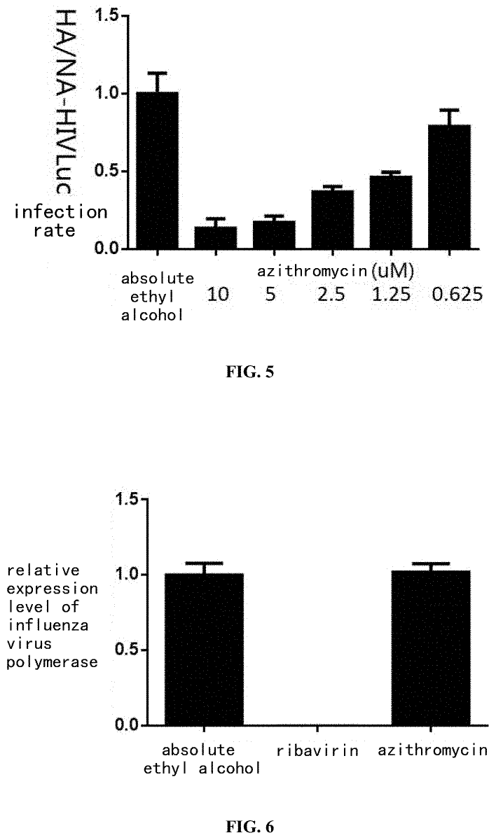

As shown in FIG. 5, after data analysis, it was shown that azithromycin inhibited influenza virus infection and the inhibitory effect was concentration-dependent.

### Example 5: Azithromycin Did not Affect the Replication of the RNA Polymerase of Influenza Virus

In 293T human renal epithelial cells, the RNA polymerase system of influenza virus was used to detect whether azithromycin would affect the transcription and replication of influenza virus by adopting the same experimental methods and steps as in Example 4. 10⁵ 293 T cells were seeded in a 24-well cell culture plate. After about 16 hours, cells were transfected with the expression plasmids of each subunit protein and NP protein of influenza virus polymerase (pcDNA-PB2, pcDNA-PB1, pcDNA-PA, and pcDNA-NP) and a plasmid that provides the RNA template of the virus (pPol-NP-luc) (5 plasmids in total) by using Lipofectamine 2000 transfection reagent, 100 ng of each of the above plasmids was transfected into cells, and an internal reference plasmid with Renilla luciferase activity was transfected at the same time. 16 hours after the transfection, absolute ethyl alcohol, azithromycin and ribavirin were added to each group in sequence, and detection and analysis were conducted after another 24 hours. Similarly, the inventors used absolute ethyl alcohol as a negative control group, and the effects of the drug on the replication of influenza virus polymerase were results relative to the result of the control group using absolute ethyl alcohol.

As shown in FIG. 6, after data analysis, it was shown that azithromycin did not affect the replication of the RNA polymerase of influenza virus.

### Example 6: Azithromycin Resisted the Influenza Virus Infection Mainly by Acting on the Virus Particles

Understanding the target and molecular mechanism of the antiviral effect of azithromycin was beneficial for the inventors to optimize the design and re-modify the drug, so as to enable the drug to produce an antiviral efficacy which was stronger and more specific. In the long run, it would also be beneficial for the inventors to further develop azithromycin as a main component of a drug with a broad-spectrum antiviral activity.

In order to get a preliminary understanding of the target of azithromycin, the inventors set up the following experiment to detect whether azithromycin exerted antiviral affect by affecting the host itself or acting on the virus particles. The inventors seeded A549 cells in a 24-well cell plate with 50000 cells per well, three groups of experiments which each comprised a control group were set up, and an absolute ethyl alcohol group and an azithromycin administration group (10 μM) were set in each group. The experimental process of the first group was as follows. Cells were pretreated with the drug at 37° C. for 2 hours, washed twice with pre-chilled PBS, then an appropriate amount of IAV-Lucinfluenza virus (A/Puerto Rico/8/34(PR8)-Luc) was added to infect the cells, and the inoculation amount of the virus was 10⁵RLU/CELL. After being cultured for 2 hours at 37° C., the resultant was washed twice with pre-chilled PBS and then placed in an incubator at 37° C., and the inhibitory effect of the drug was detected after 24 hours. The experimental process of the second group was as follows. The drug and the virus were pre-incubated at 37° C. for 2 hours and the resultant was added to A549 cells. After being cultured at 37° C. for 2 hours, the mixture was washed twice with pre-chilled PBS and then placed in an incubator at 37° C., and the inhibitory effect of the drug was detected after 24 hours. The experimental process of the third group was as follows. 2 hours after the cells were infected with influenza virus at 37° C., the resultant was washed twice with pre-chilled PBS, and then the drug was added to treat the mixture at 37° C. for 2 hours. The resultant was washed twice with PBS and then placed in an incubator at 37° C., and the inhibitory effect of the drug was detected after 24 hours. Absolute ethyl alcohol was used as a negative control group in each group of experiments, the viral infection rates achieved under different drug treatments were all results relative to the result of the absolute ethyl alcohol group.

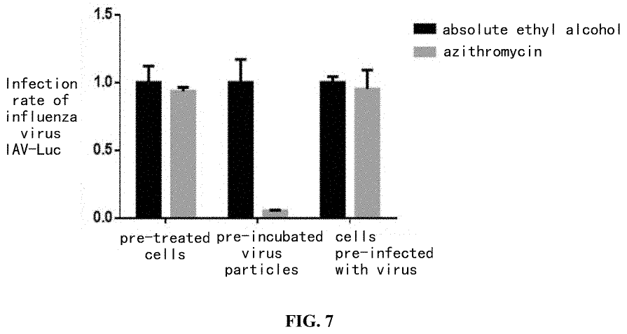

As shown in FIG. 7, azithromycin exerted antiviral effect mainly by acting on the virus particles.

The above-mentioned Examples of the present disclosure are only examples for clearly illustrating the present disclosure, and are not limitation to the embodiments of the present disclosure. For those of ordinary skill in the art to which the present disclosure pertains, it is also possible to make other changes or modifications in different forms based on the above description. There is no need to exhaustively list all the embodiments and it is also impossible to exhaustively list all the embodiments. Any modification, equivalent replacement, improvement and the like made within the spirit and principle of the present disclosure shall be included in the protection scope of the claims of the present disclosure.

### REFERENCES

1. Salomon R, Webster R G (2009) The influenza virus enigma. Cell 136:402-410.
2. Tumpey T M, Belser J A (2009) Resurrected pandemic influenza viruses. Annu Rev Microbiol 63:79-98.
3. Stamboulian D, Bonvehi P E, Nacinovich F M, Cox N (2000) Influenza. Infect Dis Clin North Am 14:141-166.
4. Hampson A W, Mackenzie J S (2006) The influenza viruses. Med J Aust 185:S39-43.
5. Du X, Dong L, Lan Y, Peng Y, Wu A, et al. (2012) Mapping of H3N2 influenza antigenic evolution in China reveals a strategy for vaccine stratin recommendation. Nat Commun 3:709.
6. De Jong J C. Beyer W E. Palache A M, Rimmlzwaan G F, Osterhaus A D (2000) Mismatch between the 1997/1998 influenza vaccine and the major epidemic A(H3N2) Virus strain as the cause of an inadequate vaccine-induced antibody response to this strain in the elderly. J Med Virol 61:94-99.
7. Arino J, Bowman C S, Moghadas S M (2009) Antiviral resistance during pandemic influenza: Implications for stockpiling and drug use. BMC Infect Dis9:8.
8. Colman P M (2009) New antiviral and drug resistance. Annu Rev Biochem 78:95-118.
9. Si Y J (2017). Genetic characterisation of novel, high pathogenic avian influenza(HPA)H5N6 viruses isolated in birds, South Korea, November 2016. Euro Surveill:22.1.30434.
10. CiminiA, dAngelo M (2017) Flavopiridol: An old drug with new perspectives? Implication for Development of New Drugs. J Cell Physiol 232(2)312-322.
11. Sancho P, Burgos-Ramos E (2015) MYC/PGC-1α balance determines the metabolic phenotype and plasticity of pancreatic cancer stem cells. Cell Metab 22(4):590-605.
12. Pathania R (2016) Combined inhibition of DNMT and HDAC blocks the tumorigrenicity of cancer stem-like cells and attenuates mammary tumor growth. Cancer Res 76(11):3224-35.
13. Morran D C (2014) Tareting mTOR dependency in pancreatic cancer. Gut 63(9):1481-9.
14. Shirakawa K (2016) Sallcylate, diflunisal and their metabolites inhibit CBP/p300 and exhibit anticancer activity. Elife. 31:5.
15. YuquangZhao (2016) Toremifene interacts with and destabilizes the Ebola virus glycoprotein. Nature. 535(7610):169-172.1
16. Delvecchio R (2016). Chloroquine, an Endocytosis blocking agent, inhibit Zika virus infection in different cell models. Viruses. 8(12):722.
17. Sakurai Y (2015). Two-pore channels control Ebola virus host cell entry and are drug targets for disease treatment. Science. 347(6225):9958.
18. M. J. Parnham (2014). Azithromycin: Mechanisms of action and their relevance for clinical application. Pharmacology & Therapeutics 143:225-245.
19. WeiqiPan (2013). Visualizing the influenza virus infection in living mice. Nat Communication 4:2369.
20. Kakeya H (2014). Efficacy of combination therapy with oseltamivir phosphate and azithromycin for influenza: a multicenter, open-label, randomized study.

What is claimed is:

1. Method of treating or preventing influenza virus infection in a subject in need thereof, the method comprising administering a macrolide antibiotic or a pharmaceutically acceptable salt thereof to said subject.

2. The method according to claim 1, wherein the antibiotic is selected from one or more of azithromycin, erythromycin, roxithromycin, midecamycin and acetyl spiramycin.

3. The method according to claim 1, wherein the antibiotic is selected from azithromycin.

4. The method according to claim 1, wherein the type of the influenza virus comprises an influenza A virus, an influenza B virus, an influenza C virus, or a combination thereof; preferably, the type of the influenza virus is the influenza A virus; more preferably, the type of the influenza virus is an influenza A H1N1 virus.

5. The method according to claim 1, wherein the drug further comprises a therapeutically effective amount of other antiviral compound(s).

6. The method according to claim 5, wherein said other antiviral compound comprises one or more of amantadine, rimantadine, oseltamivir and zanamivir.

7. The method according to claim 6, wherein the drug further comprises one or more pharmaceutically acceptable carriers.

8. The method according to claim 1, wherein the drug is administered via one of the following routes: intravenous administration, intraperitoneal administration, intracoronary administration, intraarterial administration, intradermal administration, subcutaneous administration, transdermal delivery, intratracheal administration, intraarticular administration, intraventricular administration, inhalation, intracerebral administration, transumbilical administration, oral administration, intraocular administration, pulmonary administration, injection via a catheter, administration via a suppository, and direct injection into a tissue.

9. The method of claim 5, wherein the drug is administered via one of the following routes: intravenous administration, intraperitoneal administration, intracoronary administration, intraarterial administration, intradermal administration, subcutaneous administration, transdermal delivery, intratracheal administration, intraarticular administration, intraventricular administration, inhalation, intracerebral administration, transumbilical administration, oral administration, intraocular administration, pulmonary administration, injection via a catheter, administration via a suppository, and direct injection into a tissue.

10. The method of claim 7, wherein the drug is administered via one of the following routes: intravenous administration, intraperitoneal administration, intracoronary administration, intraarterial administration, intradermal administration, subcutaneous administration, transdermal delivery, intratracheal administration, intraarticular administration, intraventricular administration, inhalation, intracerebral administration, transumbilical administration, oral administration, intraocular administration, pulmonary administration, injection via a catheter, administration via a suppository, and direct injection into a tissue

11. Use of a composition in the preparation of a drug for preventing or treating influenza virus infection, wherein the composition comprises a macrolide antibiotic or a pharmaceutically acceptable salt thereof as an active component of a drug that inhibits the influenza virus infection.

12. The use according to claim 11, wherein the antibiotic is selected from one or more of azithromycin, erythromycin, roxithromycin, midecamycin and acetylspiramycin; preferably, the antibiotic is selected from azithromycin; optionally, the composition further comprises a pharmaceutically acceptable carrier.
